(19) United States
(12) Patent Application Publication
(10) Pub. No.: US 2026/0034333 A1
(43) Pub. Date: Feb. 5, 2026

(54) MEDICAL DEVICE PACKAGING

(71) Applicant: Boston Scientific Medical Device Limited, Galway (IE)

(72) Inventors: Shoeb Khan, Nagpur (IN); Anshul Chabra, New Delhi (IN); Prateek Gupta, Gurgaon (IN)

(73) Assignee: Boston Scientific Medical Device Limited, Galway (IE)

(21) Appl. No.: 19/286,319

(22) Filed: Jul. 31, 2025

Related U.S. Application Data
(60) Provisional application No. 63/677,465, filed on Jul. 31, 2024.

(51) Int. Cl. A61M 25/00

(52) U.S. Cl. CPC A61M 25/002

(57) ABSTRACT

Packages for medical devices are disclosed. An example package may include base having a medical device holding channel formed therein. A cover may be disposed over the base. The cover may only partially overlie the medical device holding channel.

### CROSS-REFERENCE TO RELATED APPLICATIONS

This application claims the benefit of priority under 35 U.S.C. § 119 of U.S. Provisional Application No. 63/677,465, filed Jul. 31, 2024, the entire disclosure of which is hereby incorporated by reference.

### TECHNICAL FIELD

The present disclosure pertains to medical devices, and methods for manufacturing medical devices. More particularly, the present disclosure pertains to medical device packaging.

### BACKGROUND

A wide variety of medical devices have been developed for medical use, for example, intravascular use. Some of these devices include guidewires, catheters, and the like. These devices may be packaged in a number of different medical device packages. Of the known medical device packages, each has certain advantages and disadvantages. There is an ongoing need to provide alternative medical device packages.

### BRIEF SUMMARY

This disclosure provides design, material, manufacturing method, and use alternatives for medical devices and/or medical device packages. A package for a medical device is disclosed. The package comprises: a base having a medical device holding channel formed therein; and a cover disposed over the base, wherein the cover only partially overlies the medical device holding channel.

Alternatively or additionally to any of the embodiments above, the medical device holding channel includes an arcuate region.

Alternatively or additionally to any of the embodiments above, the cover has a lip that extends partially over the arcuate region.

Alternatively or additionally to any of the embodiments above, the arcuate region has a width and wherein the lip extends across 60% or less of the width.

Alternatively or additionally to any of the embodiments above, the arcuate region has a width and wherein the lip extends across 50% or less of the width.

Alternatively or additionally to any of the embodiments above, the arcuate region has a width and wherein the lip extends across 40% or less of the width.

Alternatively or additionally to any of the embodiments above, the base includes a hub region.

Alternatively or additionally to any of the embodiments above, the hub region is continuous with the medical device holding channel.

Alternatively or additionally to any of the embodiments above, the cover partially overlies the hub region.

Alternatively or additionally to any of the embodiments above, the hub region is free of the cover.

Alternatively or additionally to any of the embodiments above, the cover includes one or more securing projections configured to secure the cover to the base.

A medical device package is disclosed. The medical device package comprises: a packaging base; wherein the packaging base has an arcuate medical device holding channel formed therein configured to receive a medical device therein; wherein the packaging base has a hub region continuous with the arcuate medical device holding channel, the hub region being configured to receive a hub of the medical device; a packaging cover disposed over the packaging base; and wherein the packaging cover includes a lip that extends partially over the arcuate medical device holding channel in order to retain the medical device within the arcuate medical device holding channel and so that a user can access the medical device within the arcuate medical device holding channel.

Alternatively or additionally to any of the embodiments above, the arcuate medical device holding channel has a width and wherein the lip extends across 60% or less of the width.

Alternatively or additionally to any of the embodiments above, the arcuate medical device holding channel has a width and wherein the lip extends across 50% or less of the width.

Alternatively or additionally to any of the embodiments above, the arcuate medical device holding channel has a width and wherein the lip extends across 40% or less of the width.

Alternatively or additionally to any of the embodiments above, the packaging cover partially overlies the hub region.

Alternatively or additionally to any of the embodiments above, the hub region is free of the packaging cover.

Alternatively or additionally to any of the embodiments above, the packaging cover includes one or more securing projections configured to secure the packaging cover to the packaging base.

A method for packaging a medical device is disclosed. The method comprises: disposing a medical device within an arcuate medical device holding channel formed in a base of a medical device package assembly; and disposing cover disposed over the base, wherein the cover has a lip that extends partially over the arcuate medical device holding channel in order to retain the medical device within the arcuate medical device holding channel and so that a user can access the medical device within the arcuate medical device holding channel.

Alternatively or additionally to any of the embodiments above, the base has a hub region continuous with the arcuate medical device holding channel, and further comprising disposing a hub of the medical device within the hub region.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is an exploded view of an example medical device package.

FIG. 2 is a top view of an example medical device package.

FIG. 3 is a partially cutaway view of an example medical device package.

FIG. 4 is a top view of an example medical device package.

FIG. 5 is a top view of an example medical device package.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Medical device packages come in a variety of different forms. One example medical device package may include a two-piece tray with a base and a cover. In such packages, the base has a channel for holding the medical device therein. The cover overlies the base (and the medical device). In use, a clinician removes the cover to access the medical device prior to its use for an intervention. For a number of reasons including the mechanical properties of the medical device, the medical device may have a tendency to spring out of the package/channel, which could take the medical device out of the sterile field and/or place the medical device into a non-sterile area or it may cause damage to the medical device. In addition, during packaging of medical devices, a packager may dispose the medical device in the channel. The medical device may have a tendency to spring out of the channel during such packaging processes, thereby complicating packaging. Disclosed herein are medical device packages that may help to reduce the likelihood that a medical device may jump/spring or inadvertently be dislodged from a package/channel. Some additional details of example medical device packages are disclosed herein.

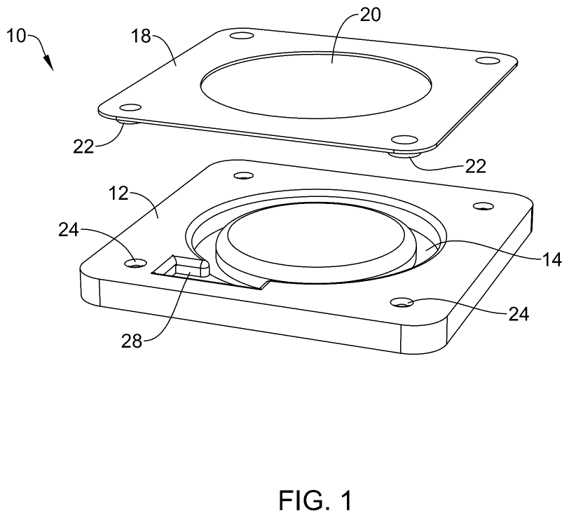

FIG. 1 is an exploded view of an example medical device package 10. The medical device package 10 may include a base or base portion 12. The base 12 may have a channel 14 formed therein. The channel 14 may have an arcuate shape or arrangement. For example, the channel 14 may be generally round in shape. Other shapes and/or arrangements are contemplated. In some instances, the base 12 may include a hub region 28. In at least some instances, the hub region 28 may be continuous with the channel 14. In other words, a medical device (not shown) such as a guidewire, a catheter, a guidewire pre-loaded in a catheter, a balloon catheter, a stent delivery system, and/or the like can be disposed with the catheter shaft in the channel 14 and the catheter hub in the hub region 28.

The medical device package 10 may also include a cover or cover portion 18. In general, the cover 18 is configured to overlie the base 12 so that a medical device can be held within therebetween (e.g., within the medical device package 10). The cover 18 may have a central opening 20. In some instances, the cover 18 may include one or more securing members or projections 22. The securing projections 22 may be configured to engage securing regions 24 formed or disposed along the base 12. For example, the securing projections 22 may be configured to fit within the securing region 24 and be secured therewith (e.g., via an interference fit, a mechanical fit/bond, a thermal bond, an adhesive bond, combinations thereof, and/or the like).

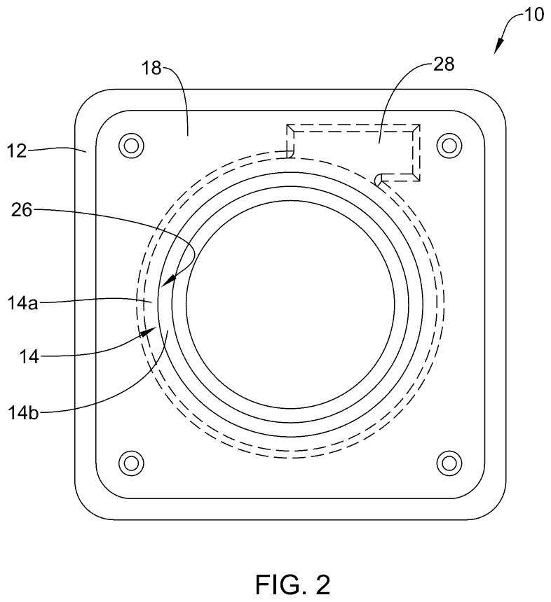

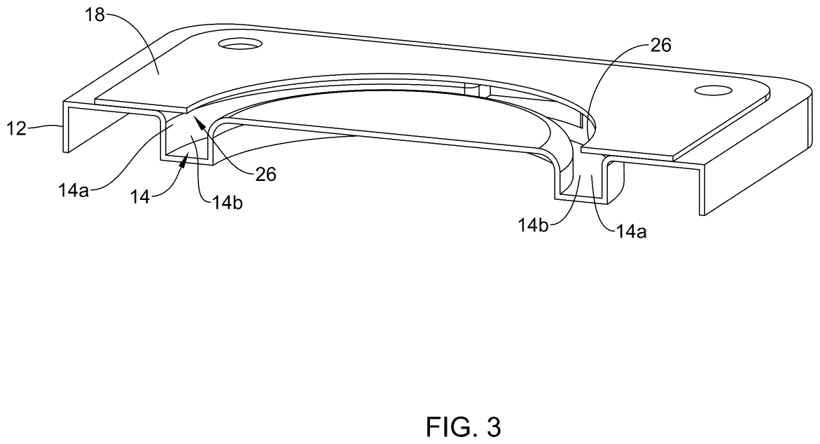

FIG. 2 illustrates the medical device package 10 in an assembled configuration. Here it can be seen that the cover 18 includes or defines a lip or lip region 26. The lip 26 partially covers the channel 14 as can be seen in FIGS. 2-3. For example, the lip 26 may divide the channel 14 into a covered portion 14a (e.g., covered by the lip 26) and an uncovered portion 14b (e.g. not covered by the lip 26). Because a portion (e.g., uncovered portion 14b) of the channel 14 is not covered, a clinician may be able to reach inside the medical device package 10, under the lip 26, and grab the medical device disposed therein and remove the medical device from the medical device package 10. Furthermore, a packager may be able to place a medical device into the channel 14 (e.g., under the lip 26) when packaging the medical device. Because a substantial portion (e.g., about 60% or less of the width of the channel 14, or about 50% or less of the width of the channel 14, or about 40% or less of the width of the channel 14) of the channel 14 may be covered by the lip 26, the lip 26 may help to secure a medical device disposed within the channel 14. This may help to reduce the likelihood that a medical device disposed within the medical device package 10 (e.g., disposed within the channel 14) may jump/spring from the medical device package 10 and/or channel 14.

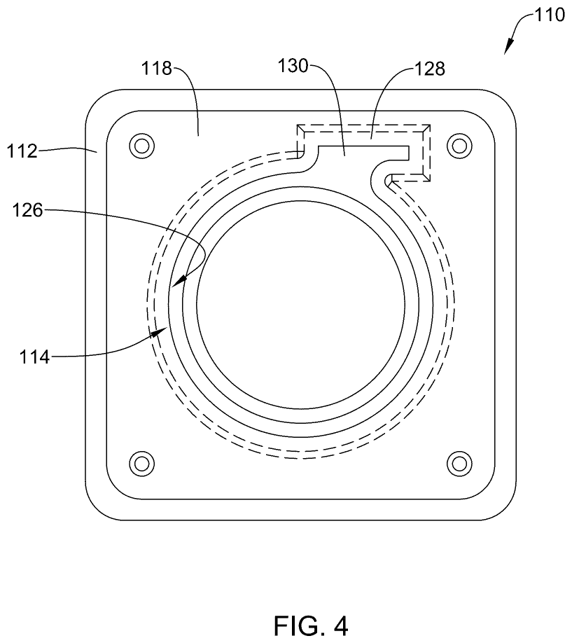

In the example medical device package 10 shown in FIGS. 1-3, the hub region 28 is covered by the cover 18. In some instances, it may be desirable for the hub region 28 to be uncovered, or be only partially covered by the cover 18. For example, FIG. 4 illustrates another example medical device package 110 that may be similar in form and function to other medical device packages disclosed herein. The medical device package 110 may include a base 112 and a cover 118. A channel 114 may be formed in the base 112. The base 112 may also include a hub region 128. The cover 118 may have a lip 126. In this example, the cover 118 may partially cover the hub region 128. For example, cover 118 may have hub lip region 130.

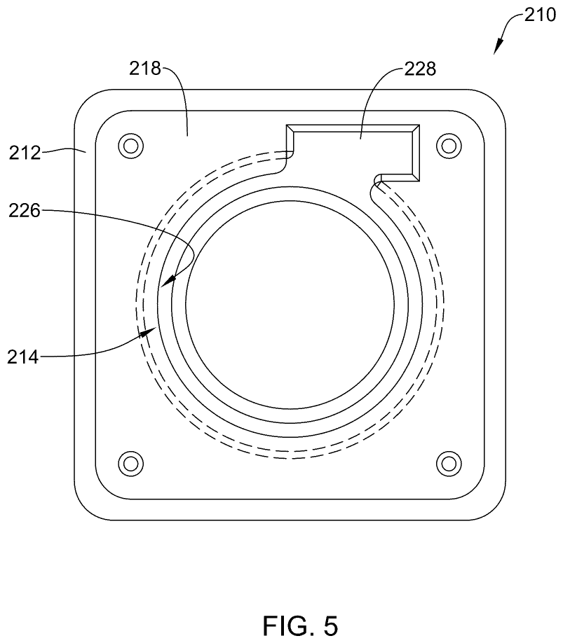

FIG. 5 illustrates another example medical device package 210 that may be similar in form and function to other medical device packages disclosed herein. The medical device package 210 may include a base 212 and a cover 218. A channel 214 may be formed in the base 212. The base 212 may also include a hub region 228. The cover 218 may have a lip 226. In this example, the cover 218 is cutaway or notched so that the hub region 228 is not covered by the cover 218.

The materials that can be used for the various medical device packages disclosed herein (and/or other the components thereof) may include those commonly associated with medical devices and/or medical device packages. For example, the medical device packages (and/or other the components thereof) may be made from polymer materials. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A package for a medical device, the package comprising:
a base having a medical device holding channel formed therein; and
a cover disposed over the base, wherein the cover only partially overlies the medical device holding channel.

2. The package of claim 1, wherein the medical device holding channel includes an arcuate region.

3. The package of claim 2, wherein the cover has a lip that extends partially over the arcuate region.

4. The package of claim 3, wherein the arcuate region has a width and wherein the lip extends across 60% or less of the width.

5. The package of claim 3, wherein the arcuate region has a width and wherein the lip extends across 50% or less of the width.

6. The package of claim 3, wherein the arcuate region has a width and wherein the lip extends across 40% or less of the width.

7. The package of claim 1, wherein the base includes a hub region.

8. The package of claim 7, wherein the hub region is continuous with the medical device holding channel.

9. The package of claim 7, wherein the cover partially overlies the hub region.

10. The package of claim 7, wherein the hub region is free of the cover.

11. The package of claim 1, wherein the cover includes one or more securing projections configured to secure the cover to the base.

12. A medical device package, comprising:
a packaging base;
wherein the packaging base has an arcuate medical device holding channel formed therein configured to receive a medical device therein;
wherein the packaging base has a hub region continuous with the arcuate medical device holding channel, the hub region being configured to receive a hub of the medical device;
a packaging cover disposed over the packaging base; and
wherein the packaging cover includes a lip that extends partially over the arcuate medical device holding channel in order to retain the medical device within the arcuate medical device holding channel and so that a user can access the medical device within the arcuate medical device holding channel.

13. The medical device package of claim 12, wherein the arcuate medical device holding channel has a width and wherein the lip extends across 60% or less of the width.

14. The medical device package of claim 12, wherein the arcuate medical device holding channel has a width and wherein the lip extends across 50% or less of the width.

15. The medical device package of claim 12, wherein the arcuate medical device holding channel has a width and wherein the lip extends across 40% or less of the width.

16. The medical device package of claim 12, wherein the packaging cover partially overlies the hub region.

17. The medical device package of claim 12, wherein the hub region is free of the packaging cover.

18. The medical device package of claim 12, wherein the packaging cover includes one or more securing projections configured to secure the packaging cover to the packaging base.

19. A method for packaging a medical device, the method comprising:
disposing a medical device within an arcuate medical device holding channel formed in a base of a medical device package assembly; and
disposing cover disposed over the base, wherein the cover has a lip that extends partially over the arcuate medical device holding channel in order to retain the medical device within the arcuate medical device holding channel and so that a user can access the medical device within the arcuate medical device holding channel.

20. The method of claim 19, wherein the base has a hub region continuous with the arcuate medical device holding channel, and further comprising disposing a hub of the medical device within the hub region.
